(19) United States
(12) Patent Application Publication
(10) Pub. No.: US 2024/0366934 A1
(43) Pub. Date: Nov. 7, 2024

(54) CANCER TREATMENT SYSTEMS WITH ENHANCED WAVEFORM GENERATING CIRCUITS

(71) Applicant: Cardiac Pacemakers, Inc., St. Paul, MN (US)

(72) Inventors: Michael J. Lyden, Shoreview, MN (US); Michael J. Kane, St. Paul, MN (US)

(73) Assignee: MAYO FOUNDATION FOR MEDICAL EDUCATION AND RESEARCH, Rochester, MN (US)

(21) Appl. No.: 18/646,084

(22) Filed: Apr. 25, 2024

Related U.S. Application Data
(60) Provisional application No. 63/464,387, filed on May 5, 2023.

(51) Int. Cl. A61N 1/36; A61N 1/02; A61N 1/05

(52) U.S. Cl. CPC A61N 1/36002; A61N 1/025; A61N 1/0529

(57) ABSTRACT

Embodiments herein relate to cancer treatment systems with electrical field generating circuits. In an embodiment, an implantable field generator device is included having a housing and a field-generating circuit disposed therein. The field-generating circuit can include a circuit ground, a positive voltage output, and a negative voltage output. An equivalent series referencing resistor is connected in series between a housing and the circuit ground. A first galvanic isolation switch and a first capacitor are connected in series between the positive voltage output and a positive electrode connection terminal. A second galvanic isolation switch and a second capacitor are connected in series between the negative voltage output and a negative electrode connection terminal. Other embodiments are also included herein.

This application claims the benefit of U.S. Provisional Application No. 63/464,387, filed May 5, 2023, the content of which is herein incorporated by reference in its entirety.

### FIELD

Embodiments herein relate to cancer treatment systems with electrical field generating circuits.

### BACKGROUND

According to the American Cancer Society, cancer accounts for nearly 25% of the deaths that occur in the United States each year. The current standard of care for cancerous tumors can include first-line therapies such as surgery, radiation therapy, and chemotherapy. Additional second-line therapies can include radioactive seeding, cryotherapy, hormone or biologics therapy, ablation, and the like. Combinations of first-line therapies and second-line therapies can also be a benefit to patients if one particular therapy on its own is not effective.

Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Various cancer therapies may have significant side effects on heathy tissue. Such side effects can vary widely depending on the type of cancer therapy but can manifest as anemia, thrombocytopenia, edema, alopecia, infections, neutropenia, lymphedema, cognitive problems, nausea and vomiting, neuropathy, skin and nail changes, sleep problems, urinary and bladder problems, and the like.

### SUMMARY

Embodiments herein relate to cancer treatment systems with electrical field generating circuits. In a first aspect, an implantable field generator device can be included having a housing and a field-generating circuit. The field-generating circuit can be disposed within the housing. The field-generating circuit can include a circuit ground, a positive voltage output, and a negative voltage output, an equivalent series referencing resistor, a first DC block, AC coupling capacitor, a first galvanic isolation switch, wherein the first galvanic isolation switch and the first DC block, AC coupling capacitor can be connected in series between the positive voltage output and a positive electrode connection terminal, a second DC block, AC coupling capacitor, and a second galvanic isolation switch, wherein the second galvanic isolation switch and the second DC block, AC coupling capacitor can be connected in series between the negative voltage output and a negative electrode connection terminal.

In a second aspect, in addition to one or more of the preceding or following aspects, or in the alternative to some aspects, the equivalent series referencing resistor can have a resistance of 20K ohms or more.

In a third aspect, in addition to one or more of the preceding or following aspects, or in the alternative to some aspects, the equivalent series referencing resistor can be configured to limit current through patient contacting therapy electrodes electrically connected with the connection terminals back to the circuit ground in the case of failure of the first DC block, AC coupling capacitor, the second DC block, AC coupling capacitor, the first galvanic isolation switch, or the second galvanic isolation switch.

In a fourth aspect, in addition to one or more of the preceding or following aspects, or in the alternative to some aspects, the first DC block, AC coupling capacitor, the second DC block, AC coupling capacitor, the first galvanic isolation switch, and the second galvanic isolation switch block DC current and low frequency AC current flow.

In a fifth aspect, in addition to one or more of the preceding or following aspects, or in the alternative to some aspects, the implantable field generator device can further include a first therapy electrode, wherein the first therapy electrode can be electrically connected with the positive electrode connection terminal, and a second therapy electrode, wherein the second therapy electrode can be electrically connected with the negative electrode connection terminal.

In a sixth aspect, in addition to one or more of the preceding or following aspects, or in the alternative to some aspects, wherein the first therapy electrode includes platinum, and wherein the second therapy electrode includes platinum.

In a seventh aspect, in addition to one or more of the preceding or following aspects, or in the alternative to some aspects, wherein the first galvanic isolation switch can be an optical isolation switch, and wherein the second galvanic isolation switch can be an optical isolation switch.

In an eighth aspect, in addition to one or more of the preceding or following aspects, or in the alternative to some aspects, the housing includes a conductive material.

In a ninth aspect, an implantable field generator can be included having a housing, a voltage supply circuit, wherein the voltage supply circuit can be disposed within the housing, and an H-bridge circuit, wherein the H-bridge circuit can be electrically connected with the voltage supply circuit, the H-bridge circuit can include a positive voltage output, and a negative voltage output, a first bandpass filter, a first capacitor, wherein the first bandpass filter and the first capacitor can be connected in series between the positive voltage output and a first supply electrode connection terminal, a second bandpass filter, a second capacitor, wherein the second bandpass filter and the second capacitor can be connected in series between the negative voltage output and a second supply electrode connection terminal, a first resistor, a second resistor, wherein the first resistor and the second resistor can be connected in series between an output of the voltage supply circuit and a ground connected to the voltage supply circuit, and a conductor, wherein the conductor provides electrical communication between the housing and a point between the first resistor and the second resistor.

In a tenth aspect, in addition to one or more of the preceding or following aspects, or in the alternative to some aspects, can further include an equivalent series referencing resistor, wherein the equivalent series referencing resistor can be connected to the conductor in series between the housing and the point between the first resistor and the second resistor.

In an eleventh aspect, in addition to one or more of the preceding or following aspects, or in the alternative to some aspects, the housing includes a conductive material.

In a twelfth aspect, in addition to one or more of the preceding or following aspects, or in the alternative to some aspects, the voltage supply circuit can have a voltage output of 0 to 12 volts DC.

In a thirteenth aspect, in addition to one or more of the preceding or following aspects, or in the alternative to some aspects, the implantable field generator can further include a first therapy electrode, wherein the first therapy electrode can be electrically connected with the first supply electrode connection terminal, and a second therapy electrode, wherein the second therapy electrode can be electrically connected with the second supply electrode connection terminal.

In a fourteenth aspect, an implantable field generator can be included having a housing, a voltage supply circuit, wherein the voltage supply circuit can be disposed within the housing, and an H-bridge circuit, wherein the H-bridge circuit can be electrically connected with the voltage supply circuit, the H-bridge circuit can include a positive voltage output, and a negative voltage output, a first LC filter, a first capacitor, wherein the first LC filter and the first capacitor can be connected in series between the positive voltage output and a first supply electrode terminal, a second LC filter, a second capacitor, wherein the second LC filter and the second capacitor can be connected in series between the negative voltage output and a second supply electrode terminal, a first resistor, a second resistor, wherein the first resistor and the second resistor can be connected in series between an output of the voltage supply circuit and a ground connected to the voltage supply circuit, a conductor, wherein the conductor can be connected between the housing and a point between the first resistor and the second resistor, and a two-phase, non-overlapping clock signal generator, wherein the two-phase, non-overlapping clock signal generator can be electrically connected with the H-bridge circuit.

In a fifteenth aspect, in addition to one or more of the preceding or following aspects, or in the alternative to some aspects, can further include vector selection relays, wherein the vector selection relays can be connected in series with a first supply electrode connection terminal and/or a second supply electrode connection terminal.

In a sixteenth aspect, in addition to one or more of the preceding or following aspects, or in the alternative to some aspects, the vector selection relays can be optically coupled solid-state relays.

In a seventeenth aspect, in addition to one or more of the preceding or following aspects, or in the alternative to some aspects, the housing includes a conductive material.

In an eighteenth aspect, in addition to one or more of the preceding or following aspects, or in the alternative to some aspects, the implantable field generator can further include a first therapy electrode, wherein the first therapy electrode can be electrically connected with a first supply electrode connection terminal, the first supply electrode connection terminal electrically connected with the first capacitor, and a second therapy electrode, wherein the second therapy electrode can be electrically connected with a second supply electrode connection terminal, the second supply electrode connection terminal electrically connected with the second capacitor.

In a nineteenth aspect, an implantable field generator can be included having a housing, a voltage supply circuit, wherein the voltage supply circuit can be disposed within the housing, and an H-bridge circuit, wherein the H-bridge circuit can be electrically connected with the voltage supply circuit, the H-bridge circuit can include a positive voltage output, and a negative voltage output, a first low pass filter, a first capacitor, wherein the first low pass filter and the first capacitor can be connected in series between the positive voltage output and a first supply electrode connection terminal, a second low pass filter, a second capacitor, wherein the second low pass filter and the second capacitor can be connected in series between the negative voltage output and a second supply electrode connection terminal, a programmable logic device, wherein the programmable logic device can be electrically connected with the H-bridge circuit, and wherein the programmable logic device includes four signal outputs received by the H-bridge.

In a twentieth aspect, in addition to one or more of the preceding or following aspects, or in the alternative to some aspects, can further include a μP interface, wherein the μP interface can be in electronic communication with the programmable logic device.

In a twenty-first aspect, in addition to one or more of the preceding or following aspects, or in the alternative to some aspects, can further include an I2C, wherein the I2C can be in electronic communication with the programmable logic device.

In a twenty-second aspect, in addition to one or more of the preceding or following aspects, or in the alternative to some aspects, the programmable logic device can have a frequency from 21 to 100 MHz.

In a twenty-third aspect, in addition to one or more of the preceding or following aspects, or in the alternative to some aspects, wherein the first low pass filter can have a cutoff frequency of about 270 kHz, and wherein the second low pass filter can have a cutoff frequency of about 270 kHz.

In a twenty-fourth aspect, in addition to one or more of the preceding or following aspects, or in the alternative to some aspects, the housing includes a conductive material.

In a twenty-fifth aspect, in addition to one or more of the preceding or following aspects, or in the alternative to some aspects, the implantable field generator can further include a first therapy electrode, wherein the first therapy electrode can be electrically connected with the first supply electrode connection terminal, and a second therapy electrode, wherein the second therapy electrode can be electrically connected with the second supply electrode connection terminal.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a medical device implanted in a patient in accordance with various embodiments herein.

FIG. 2 is a schematic view of the placement of various cancer therapy leads in a region of tumor resection in accordance with various embodiments herein.

FIG. 3 is a schematic cross-sectional view of a medical device in accordance with various embodiments herein.

FIG. 4 is a schematic circuit diagram illustrating aspects in accordance with various embodiments herein.

FIG. 5 is a schematic circuit diagram illustrating aspects in accordance with various embodiments herein.

FIG. 6 is a schematic circuit diagram illustrating aspects in accordance with various embodiments herein.

FIG. 7 is a schematic circuit diagram illustrating aspects in accordance with various embodiments herein.

FIG. 8 is a schematic circuit diagram illustrating aspects in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Cancer therapies including the application of electrical fields configured to prevent and/or disrupt cellular mitosis of cancerous cells are a new approach to cancer treatment which offer the potential benefit of greatly reduced negative side effects. Such systems/device include electric field generation circuitry. However, it can be desirable when designing electrical field generation circuitry to prevent unwanted/harmful low frequency current flow through therapy electrodes that could cause neurostimulation generally, as well as in the specific scenarios of turning ON or OFF therapy output or adjusting the therapy output higher or lower. In addition, it can be desirable to prevent the forward bias of field generation circuitry and associated malfunction and well as prevent unwanted defibrillation and electrocautery current flow through therapy electrodes. Another goal for the design of field generation circuitry is the reduction of output filter requirements, and associated inductor losses, as well as internal field generator heating. Various of these goals as well as others can be achieved with embodiments of field generation devices and associated circuits herein.

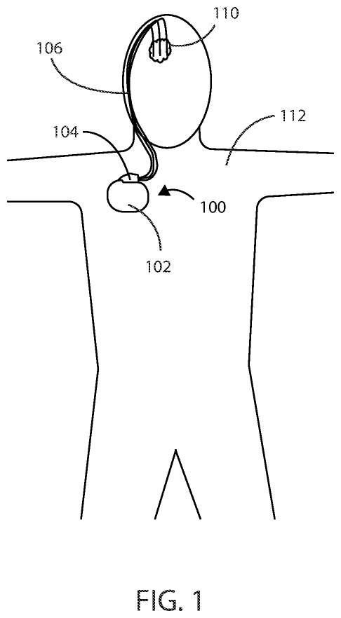

Referring now to FIG. 1, a schematic view of a medical device 100 implanted in a patient 112 is shown in accordance with the embodiments herein. In FIG. 1, the patient 112 has the medical device 100 implanted entirely within the body of the patient 112 at or near a tumor resection site 110. It will be appreciated that while many embodiments herein disclose a tumor resection site 110, area 110 may alternatively represent a cancerous or non-cancerous tumor site, zone, or cavity. Various implant sites can be used including areas such as in the limbs, the upper torso, the abdominal area, the head, and the like. In some embodiments, the medical device can be at least partially implanted within the body of the patient at or near the site of the cancerous tumor.

The medical device 100 can include a housing 102 and a header 104 coupled to the housing 102. Various materials can be used. However, in some embodiments, the housing 102 can be formed of a material such as a metal, ceramic, polymer, composite, or the like. In some embodiments, the housing 102 and/or portions thereof can specifically be formed of a conductive material. In some embodiments, the housing 102, or one or more portions thereof, can be formed of titanium. The header 104 can be formed of various materials, but in some embodiments the header 104 can be formed of a translucent polymer such as an epoxy material. In some embodiments the header 104 can be hollow. In other embodiments the header 104 can be filled with components and/or structural materials such as epoxy or another material such that it is non-hollow.

In some embodiments where a portion of the medical device 100 is partially external, the header 104 and housing 102 can be surrounded by a protective casing made of durable polymeric material. In other embodiments, where a portion of the medical device 100 is partially external, the header 104 and housing 102 can be surrounded by a protective casing made of a combination of polymeric material, metallic material, and/or glass material.

Header 104 can be coupled to one or more leads, such as leads 106. The header 104 can serve to provide fixation of the proximal end of one or more leads 106 and electrically couple the one or more leads 106 to one or more components within the housing 102. As such, one or more connection terminals, contacts, and/or pins can be disposed within the header 104.

The one or more leads 106 can include one or more electrodes (not shown in this view) disposed along the length of the leads 106. In some embodiments, electrodes can include supply electrodes, also referred to herein as “electric field therapy supply electrodes.” In some embodiments electrodes can include electric field sensing electrodes, also referred to herein as “sensing electrodes.” In some embodiments, leads 106 can include both supply electrodes and sensing electrodes. In other embodiments, leads 106 can include any number of electrodes that are both supply electrodes and sensing electrodes.

The one or more leads 106 can also include one or more temperature sensors (not shown in this view) disposed along the length of the leads 106. Temperature sensors herein can include, but are not limited to, various types of optical and electrical temperature sensors. Temperature sensors herein can include contact-type temperature sensors and non-contact type temperature sensors. Optical temperature sensors herein can include infrared optical temperature sensors. Some optical temperature sensors can measure temperature at a distance such as a distance of millimeters or centimeters. Thus, even where temperature sensors are mounted along a lead 106, temperature can be measured at a distance therefrom. Exemplary electrical temperature sensors can include, but are not limited to, thermistors, resistive temperature detectors, thermocouples, semiconductor based temperature sensors, and the like.

In some embodiments, the medical device system can include a temperature sensor disposed remotely from the medical device. A remote temperature sensor can provide temperature data in addition to or in replace of temperature sensors in other areas such as along the leads 106. In some embodiments, a remote temperature sensor can be used to gather a core or reference temperature of the patient into which the system is implanted.

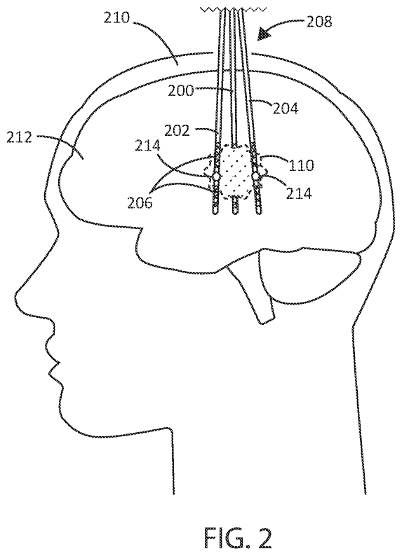

In some embodiments, the medical device can include a plurality of therapy leads implanted at or near a site a cancerous tumor or tumor resection. Referring now to FIG. 2, a schematic view of a placement of various cancer therapy leads 200, 202, 204 in a region of a tumor resection site 110 or tumor is shown in accordance with various embodiments herein. In the example of FIG. 2, temperature sensors 214 are disposed on the therapy leads. The temperature sensors 214 can be of any type described elsewhere herein. In the embodiment shown in FIG. 2, each cancer therapy lead includes two or more supply electrodes 206 disposed along a length of the cancer therapy leads. Each cancer therapy lead includes a proximal and a distal supply electrode.

The side view shown in FIG. 2 also includes the placement of cancer therapy leads 200, 202, and 204 around the tumor resection site 110 and in position within a burr hole 208 entry point on the patient's skull 210 within the patient's brain 212. It will be appreciated that in some embodiments one burr hole can be used with one or more (e.g., one, two, three, or more) leads and/or electrodes. In some embodiments, multiple burr holes can be used each with one or more (e.g., one, two, three, or more) leads and/or electrodes.

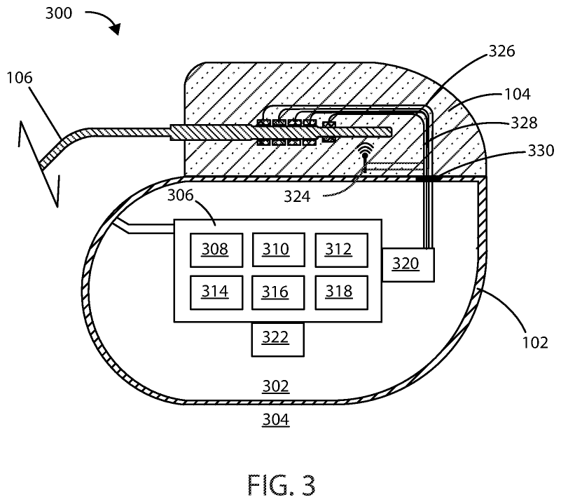

Referring now to FIG. 3, a schematic view of aspects exemplary medical device 300 is shown in accordance with various embodiments herein. The housing 102 can define an interior volume 302 that can be hollow and that in some embodiments is hermetically sealed off from the area 304 outside of medical device 300. In other embodiments the housing 102 can be filled with components and/or structural materials such that it is non-hollow. The medical device 300 can include control circuitry 306, which can include various components 308, 310, 312, 314, 316, and 318 disposed within housing 102. In some embodiments, these components can be integrated and in other embodiments these components can be separate. In yet other embodiments, there can be a combination of both integrated and separate components. The medical device 300 can also include an antenna 324, to allow for unidirectional or bidirectional wireless data communication, such as with an external device or an external power supply. In some embodiments, the components of medical device 300 can include an inductive energy receiver coil (not shown) communicatively coupled or attached thereto to facilitate transcutaneous recharging of the medical device via recharging circuitry.

The various components 308, 310, 312, 314, 316, and 318 of control circuitry 306 can include, but are not limited to, a microprocessor, memory circuit (such as random access memory (RAM), read only memory (ROM)) and/or Electrically Erasable ROM (EEROM/Flash), recorder circuitry, controller circuit, a telemetry circuit, a power supply circuit (such as a battery), a timing circuit, and an application specific integrated circuit (ASIC), a recharging circuit, amongst others. Control circuitry 306 can be in communication with electric field generating circuitry 320 that can be configured to generate electric current to create one or more fields. The electric field generating circuitry 320 can be integrated with the control circuitry 306 or can be a separate component from control circuitry 306. Control circuitry 306 can be configured to control delivery of electric current from the electric field generating circuit 320. Details of the electric field generating circuitry 320 are described in greater detail below.

In various embodiments, one or more leads 106 can be in electrical communication with the electric field generating circuit 320. The one or more leads 106 can include one or more electrodes. In some embodiments, various electrical conductors, such as electrical conductors 326 and 328, can pass from the header 104 through a feed-through structure 330 and into the interior volume 302 of medical device 300. As such, the electrical conductors 326 and 328 can serve to provide electrical connection between the one or more leads 106 and control circuitry 306 disposed within the interior volume 302 of the housing 102. In some embodiments, the control circuitry 306 can be programmed and electronically configured to direct the electric field generating circuit 320 to deliver an electric field via lead(s) 106 distal electrodes and/or the housing 102 to the site of a targeted tissue for therapy (e.g., a cancerous tumor located within a bodily tissue, a tumor resection site, or another area targeted for therapy).

In some embodiments, medical device 300 can include an electric field sensing circuit 322 configured to generate a signal corresponding to sensed electric fields. Electric field sensing circuit 322 can be integrated with control circuitry 306 or it can be separate from control circuitry 306. Sensing electrodes can be disposed on or adjacent to the housing of the medical device, on one or more leads connected to the housing, on a separate device implanted near or in the tumor, or any combination of these locations. In some embodiments, the electric field sensing circuit 322 can measure the electrical potential difference (voltage) between a first electrode and a second electrode, wherein the first and second electrodes are in any of the aforementioned locations. In some embodiments, the electric field sensing circuit can be configured to measure sensed electric fields and to record electric field strength in e.g., V/cm.

In some embodiments, recorder circuitry can be configured to record the data produced by the electric field sensing circuit 322 and record time stamps regarding the same. In some embodiments, the control circuitry 306 can be hardwired to execute various functions, while in other embodiments the control circuitry 306 can be directed to implement instructions executing on a microprocessor or other external computation device. A wireless communication interface can also be provided for communicating with external computation devices such as a programmer, a home-based unit, and/or a mobile unit (e.g., a cellular phone, personal computer, smart phone, tablet computer, smartwatch, and the like).

It will be appreciated that low frequency current flow through therapy electrodes has the potential for causing neurostimulation. Embodiments herein can prevent low frequency current flow through therapy electrodes that could cause neurostimulation. Various embodiments herein can include a field-generating circuit with a ground that is DC referenced to a metal of the supply electrode (such as platinum) via a single reference resistor to the housing. By having a single DC connection between the medical device internal circuitry (300) and patient contacting electrodes and device case, undesired DC or low frequency current flow, such as between patient therapy electrodes and/or the housing, will not occur. Also, in various embodiments, a high value resistor (such as 100K ohms or more) can be used to limit unintentional current if one of the DC blocking therapy capacitors were to fail (“short” or become “leaky”).

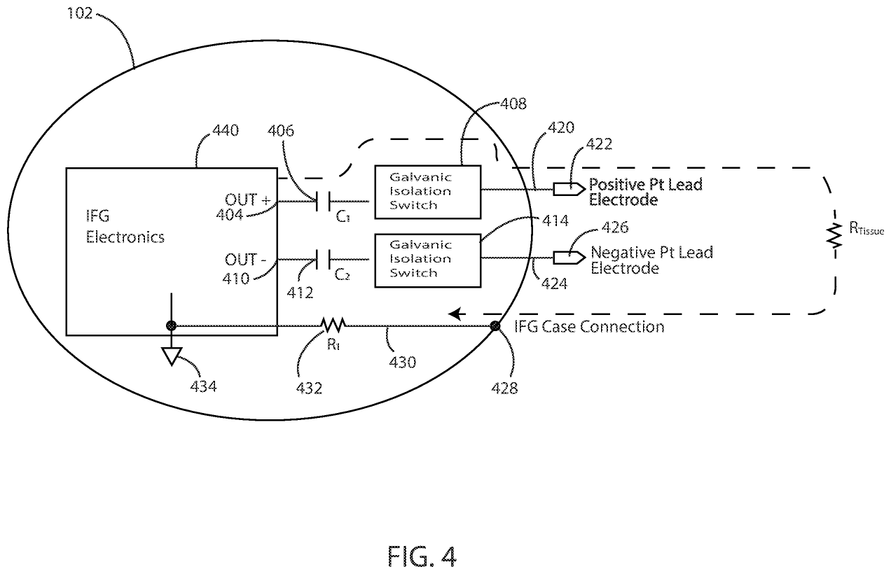

For example, referring now to FIG. 4, a schematic circuit diagram is shown in accordance with various embodiments herein. An implantable field generator device herein includes a housing 102 and electronics disposed therein. For example, the implantable field generator device includes a field-generating circuit 440. The field-generating circuit 440 includes a positive voltage output 404 and a negative voltage output 410. The field-generating circuit 440 also includes a circuit ground 434.

The implantable field generator device includes a first capacitor 406 and first galvanic isolation switch 408 in series with the positive voltage output 404. The implantable field generator device also includes a second capacitor 412 and a second galvanic isolation switch 414 in series with the negative voltage output 410. In various embodiments, the first galvanic isolation switch 408 and the second galvanic isolation switch can be optical isolation switches, however other types of switches are contemplated herein.

The first galvanic isolation switch 408 can electrically connect to a first therapy lead conductor 420, and therapy electrode 422 to the positive voltage output 404 through 406. Similarly, the second galvanic isolation switch 414 can electrically connect to a second therapy lead conductor 424. The first therapy lead conductor 420 can be electrically connected to a first therapy electrode 422. In various embodiments, the first therapy electrode 422 include platinum. The second therapy lead conductor 424 can be in electrical communication with a second therapy electrode 426. In various embodiments, the second therapy electrode 426 can include platinum.

In the example of FIG. 4, a single patient contacting electrode (the housing 102) provides field-generating circuit with a DC galvanic conduction current path to the patient. The housing 102 can include a housing contact 428 or connection point with a conductor 430. In various embodiments, the housing 102 is formed of a conductive material, such as a biocompatible metal. The implantable field generator device can also include an equivalent series referencing resistor 432 connected via the conductor 430 to the housing contact 428 and the circuit ground 434. In various embodiments, the equivalent series referencing resistor 432 can be in electrical communication in series between a housing 102 and a circuit ground 434. In various embodiments, the equivalent series referencing resistor 432 has a resistance of 10K, 50K, or 100K ohms or more. In various embodiments, the equivalent series referencing resistor 432 can be configured to limit current from therapy electrodes in electrical contact with the connection terminals back to a circuit ground 434 in the case of failure of a first capacitor 406, a second capacitor 412, a first galvanic isolation switch 408, or a second galvanic isolation switch 414.

In various embodiments herein, therapy AC coupling capacitors and/or galvanically isolated switches can be used to block DC and low frequency current flow through patient tissues. In various embodiments, the first capacitor 406, the second capacitor 412, the first galvanic isolation switch 408, and the second galvanic isolation switch 414 block DC current and low frequency AC current flow.

In some embodiments, output therapy can be AC coupled through series connected DC blocking capacitors to the patient electrodes preventing DC and low frequency current flow. This can be used in embodiments herein as an approach to prevent low frequency current flow through therapy electrodes that could cause neurostimulation.

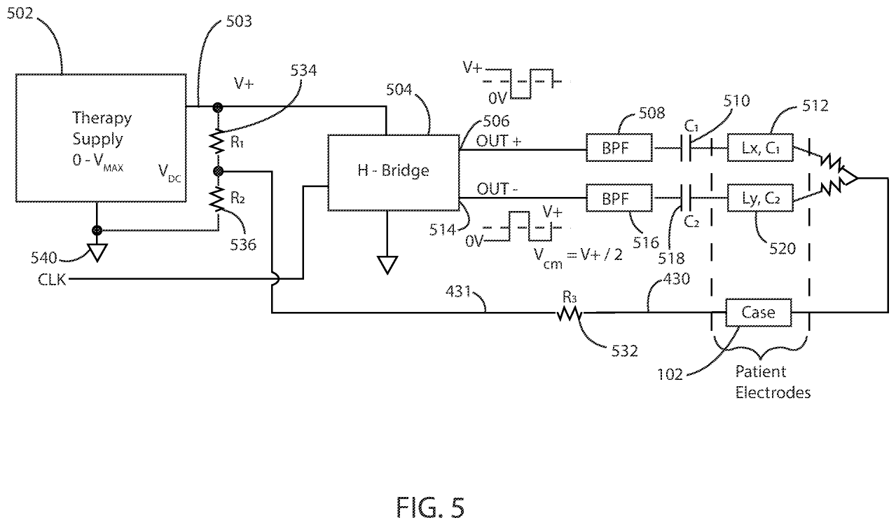

Referring now to FIG. 5, a schematic circuit diagram is shown in accordance with various embodiments herein. The implantable field generator includes a voltage supply circuit 502 with a voltage output 503 connected to an H-bridge circuit 504. The H-bridge circuit 504 includes a positive voltage output 506 and a negative voltage output 514. The positive voltage output 506 is connected to a first bandpass filter 508, which is in turn connected to a first capacitor 510. The opposite terminal of the first capacitor 510 is electrically connected to a first therapy lead 512. The negative voltage output 514 is connected to a second bandpass filter 516, which is in turn connected to a second capacitor 518. The opposite terminal of the second capacitor 518 is electrically connected to a second therapy lead 520.

The housing 102 can be connected to a resistor 532 via a conductor 430. A first resistor 534 and a second resistor 536 connected in series between a voltage output 503 of the voltage supply circuit 502 and a ground 540 forming a resistor voltage divider whose output at 431 approximate the average common mode DC voltage expected at BPF, 508, 526 outputs. As the common mode voltage is applied to IFG case, it balances/matches the CM bias on the left hand side of C1 and C2 suppressing transient low frequency current flow across the blocking capacitor and distal lead electrode when turning on, off, or otherwise adjusting the therapy output supply. connected to the voltage supply circuit 502. The conductor 430 can provide a connection between the resistor 532 and a point between the first resistor 534 and second resistor 536.

In various embodiments, the voltage supply circuit 502 has a voltage output of 0 to 34 volts DC, however various voltage output values are contemplated herein.

In various embodiments, an H-bridge circuit can be used to produce a differential output that when AC coupled to the therapy supply electrodes provides balanced fully differential electrode voltage with respect to the housing (which is equivalent to the global patient body potential). The H-bridge drive effectively doubles the available output voltage available from the therapy power supply especially needed for higher therapy tissue impedance. The balanced AC coupled drive aspect ensures that average values of the sinusoidal output voltage at the distal electrodes with respect to housing/patient body potential is zero. Similarly, the average voltage difference between therapy electrodes in the brain are also zero.

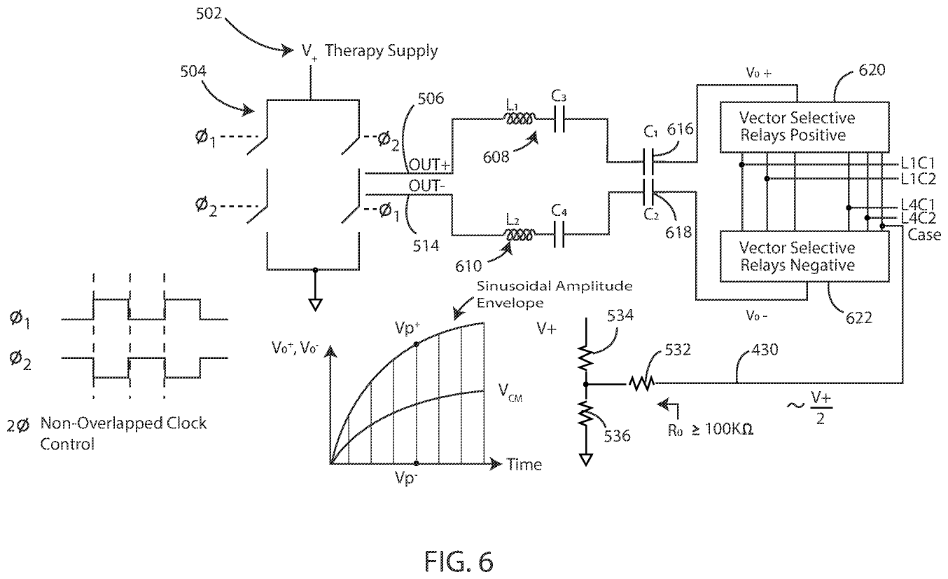

Referring now to FIG. 6, a schematic circuit diagram is shown in accordance with various embodiments herein. The implantable field generator includes a voltage supply circuit output 502 connected to an H-bridge circuit 504. A two-phase, non-overlapping clock signal generator can drive and control the four transistors of the H-bridge circuit 504.

The H-bridge circuit 504 includes a positive voltage output 506 and a negative voltage output 514. The positive voltage output 506 is connected to a first LC filter 608. The negative voltage output 514 is connected to a second LC filter 610. The first LC filter 608 is AC coupled to the positive output select relays 620 by DC blocking capacitor 616. The second LC filter 610 is AC coupled to the negative output select relays 622 by DC blocking capacitor 618. Series resonant LC bandpass filters 616, and 618 pass sinusoidal output current at the H-bridge square wave output voltage drive fundamental frequency, f₀, while suppressing its higher odd harmonics, f₃, f₅, f₇ . . . etc.

Vector selection relays can be included to provide selective connection between the therapy generation circuitry and various electrodes disposed on one or more therapy supply leads. In this example, the vector selection relays include a set of positive (Anode) output therapy relays 620 and a set of negative (Cathode) relays 622. The vector selection relay blocks can connect device therapy to patient tissue contacting electrodes on the therapy leads and/or CAN (or conductive device housing) to either positive or negative polarity as desired to control the direction and amplitude of the electric fields between therapy electrodes. In various embodiments, the vector selection relays are optically coupled solid-state relays. However, various types of relays are contemplated herein.

As before, the housing can be connected to a resistor voltage divider, resistor 534 and 536 through a current limiting resistor 532 via a conductor 430 to establish the device DC case potential to be nearly the same as the average common mode potential DC as the left hand side of C3 and C4.

In various embodiments, an H-bridge circuit with square wave drive can be utilized followed by series resonant LC filters bandpass filter out the high frequency odd harmonics of the square wave. Lower values of inductance tended to have lower losses. However, the smallest value of inductance must be large enough though that stray inductance (e.g., patient lead inductance does not significantly affect resonant band-pass tuning at the therapy output frequency, e.g. 200 KHz).

In various embodiments, systems herein can be configured to prevents forward bias of field generator circuitry and associated malfunction as well as unwanted/harmful defibrillation and electrocautery current flow through therapy electrodes that could cause tissue damage and or neurostimulation. This can be implemented in various ways. As one specific example, in some embodiments optically coupled solid state relays (such as optically coupled floating gate back-back connected NMOS output transistors) with >500 Vrms isolation can be used to ensure external defibrillator, electrosurgery equipment are not able to forward bias IFG electronics and drive harmful current through the patient electrodes when they are disabled. In some embodiments, the device can be configured to receive a command or commands (therapy OFF command, electrocautery mode command, and MRI mode command, etc.) that when communicated to the device cause opening of the relays.

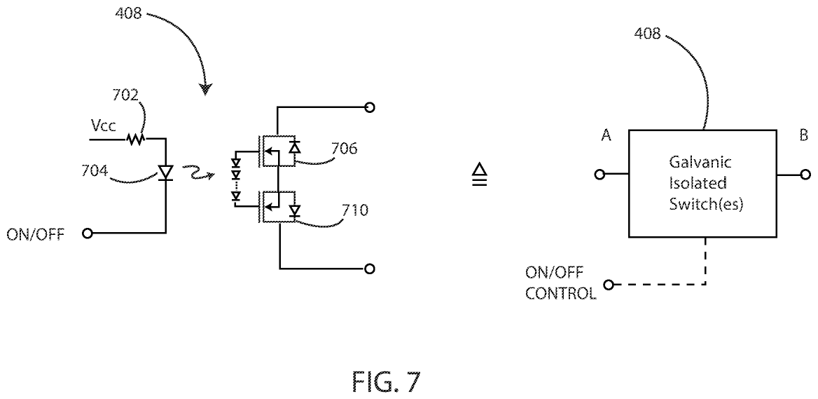

Referring now to FIG. 7, a schematic circuit diagram is shown in accordance with various embodiments herein. In specific, FIG. 7 shows a circuit diagram of an exemplary galvanic isolation switch herein. The galvanic isolation switch includes a first resistor 702 and an LED 704. The first galvanic isolation switch 408 also includes floating gate back-back connected NMOS output transistors 706, 710 which when illuminated by the optically coupled LED control the state of the switch, open or closed.

Pulse Width Modulated (PWM) H-bridge Class D generation of therapy output waveforms can be desirable over square wave drive modulation to reduce filtering requirements, inductor sizes, and associated wire and AC core losses. Conventional Class-D PWM approaches use a triangle wave carrier modulation/sampling approach whereby a high frequency carrier is “modulated” by the envelope of the waveform that is to be amplified/generated. In embodiments herein, a carrier frequency of ˜20× the frequency of the 200 KHz sinusoidal therapy output waveform is desired. Specifically, 21× can be chosen because it suppresses odd harmonics, especially the 3rd of the resulting modulated waveform, thereby reducing filtering requirements. However, triangle wave modulation front end used to amplify/produce a general signal, e.g. audio, are not needed for therapy applications herein, which may utilize a fixed 200 Khz sinewave.

In embodiments herein, periodic PWM H-bridge drive signals can be generated off-line using conventional triangle wave sampling mathematics. However, once the H-bridge clock signal sequence for generating the 200 KHz sinewave are determined, the 4 H-bridge clock controls can be provided using a programmable logic device PLD (e.g. FPGA, CPLD, etc.) to “play-back” the 4 H-bridge clock outputs, in a cyclical logic 0, 1 pattern to produce the 200 KHz sine wave.

The output of the H-bridge can then be filtered using a simple LC low-pass filter to remove the PWM switching harmonics. The low pass inductor value and associated wire and AC core loss are minimized compared to series resonant inductor losses of a square wave drive approach. Inductor AC losses can be much lower than the series resonant LC filter (˜10%) using the alternative square wave drive method which require all of the therapy output current to pass through the inductor.

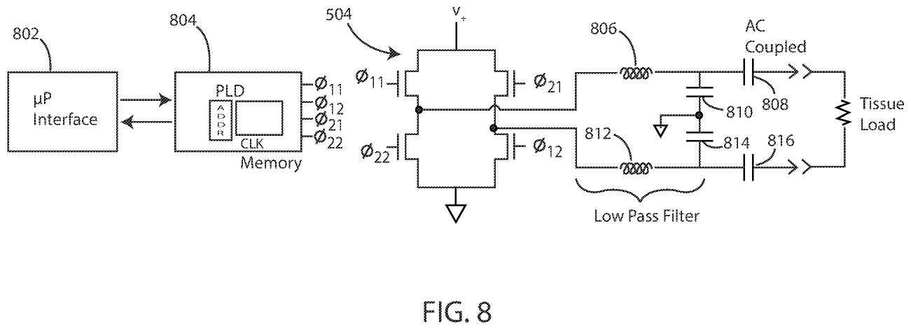

Referring now to FIG. 8, a schematic circuit diagram is shown in accordance with various embodiments herein. The implantable field generator can include an industry standard u P interface 802, such as an I2C, or SPI. In various embodiments, the uP interface 802 can be in electronic communication with a programmable logic device 804. In various embodiments, the programmable logic device 804 has a frequency from 21 to 100 MHz. However, other frequencies are contemplated herein.

In various embodiments, the programmable logic device 804 can be electrically connected to an H-bridge circuit 504. In various embodiments, the programmable logic device 804 includes four signal outputs received by the H-bridge. A first low pass filter can be applied to the positive output of the H-bridge circuit. In some embodiments, the first low pass filter can include a first inductor 806 and a first filter capacitor 810 arranged as shown in FIG. 8. Similarly, a second low pass filter can be applied to the negative output of the H-bridge circuit. In some embodiments, the second low pass filter can include a second inductor 812 and a second filter capacitor 814 arranged as shown in FIG. 8. The outputs of these LPGs are in turn AC coupled (DC blocked) by capacitors 808 and 816 before the outputs are coupled to the tissue electrodes.

In various embodiments, the first low pass filter has a cutoff frequency of about 270 kHz and the second low pass filter has a cutoff frequency of about 270 kHz. However, other specific cutoff frequencies are also contemplated herein commensurate with the output therapy frequency.

### Methods

Many different methods are contemplated herein, including, but not limited to, methods of making, methods of using, methods of generating electrical fields for therapy herein, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

### Electric Field Therapy Parameters

In some embodiments, medical devices herein can generate one or more electric fields at frequencies selected from a range of between 10 kHz to 1 MHz. In some embodiments, the one or more electric fields can be effective to prevent and/or disrupt cellular mitosis in a cell. In some embodiments, the one or more electric fields can be effective to prevent and/or disrupt cellular mitosis in a cell, but not cause tissue ablation. In some embodiments, the system can be configured to deliver an electric field at one or more frequencies selected from a range of within 300 kHz to 500 kHz. In some embodiments, the system can be configured to deliver an electric field at one or more frequencies selected from a range of within 100 kHz to 300 kHz. In some embodiments, the system can be configured to periodically deliver an electric field using one or more frequencies greater than 10 KHz.

A desired electric field strength can be achieved by delivering an electric current between two electrodes. The specific current and voltage at which the electric field is delivered can vary and can be adjusted to achieve the desired electric field strength at the site of the tissue to be treated. In some embodiments, the system can be configured to deliver an electric field using currents ranging from 1 mAmp to 1000 mAmp to the site of a cancerous tumor. In some embodiments, the system can be configured to deliver an electric field using currents ranging from 20 mAmp to 500 mAmp to the site of a cancerous tumor. In some embodiments, the system can be configured to deliver an electric field using currents ranging from 30 mAmp to 300 mAmp to the site of a cancerous tumor.

In some embodiments, the system can be configured to deliver an electric field using currents including 1 mAmp, 2 mAmp, 3 mAmp, 4 mAmp, 5 mAmp, 6 mAmp, 7 mAmp, 8 mAmp, 9 mAmp, 10 mAmp, 15 mAmp, 20 mAmp, 25 mAmp, 30 mAmp, 35 mAmp, 40 mAmp, 45 mAmp, 50 mAmp, 60 mAmp, 70 mAmp, 80 mAmp, 90 mAmp, 300 mAmp, 125 mAmp, 150 mAmp, 175 mAmp, 400 mAmp, 225 mAmp, 250 mAmp, 275 mAmp, 300 mAmp, 325 mAmp, 350 mAmp, 375 mAmp, 400 mAmp, 425 mAmp, 450 mAmp, 475 mAmp, 500 mAmp, 525 mAmp, 550 mAmp, 575 mAmp, 600 mAmp, 625 mAmp, 650 mAmp, 675 mAmp, 700 mAmp, 725 mAmp, 750 mAmp, 775 mAmp, 800 mAmp, 825 mAmp, 850 mAmp, 875 mAmp, 900 mAmp, 925 mAmp, 950 mAmp, 975 mAmp, or 1000 mAmp. It will be appreciated that the system can be configured to deliver an electric field at a current falling within a range, wherein any of the forgoing currents can serve as the lower or upper bound of the range, provided that the lower bound of the range is a value less than the upper bound of the range.

In some embodiments, the system can be configured to deliver an electric field using voltages ranging from 1 Vrms to 50 Vrms to the site of a cancerous tumor. In some embodiments, system can be configured to deliver an electric field using voltages ranging from 5 Vrms to 30 Vrms to the site of a cancerous tumor. In some embodiments, the system can be configured to deliver an electric field using voltages ranging from 10 Vrms to 20 Vrms to the site of a cancerous tumor.

In some embodiments, the system can be configured to deliver an electric field using one or more voltages including 1 Vrms, 2 Vrms, 3 Vrms, 4 Vrms, 5 Vrms, 6 Vrms, 7 Vrms, 8 Vrms, 9 Vrms, 10 Vrms, 15 Vrms, 20 Vrms, 25 Vrms, 30 Vrms, 35 Vrms, 40 Vrms, 45 Vrms, or 50 Vrms. It will be appreciated that the system can be configured to deliver an electric field at a voltage falling within a range, wherein any of the forgoing voltages can serve as the lower or upper bound of the range, provided that the lower bound of the range is a value less than the upper bound of the range.

In some embodiments, the system can be configured to deliver an electric field using one or more frequencies including 10 kHz, 20 kHz, 30 kHz, 40 kHz, 50 kHz, 60 kHz, 70 kHz, 80 kHz, 90 kHz, 300 kHz, 125 kHz, 150 kHz, 175 kHz, 400 kHz, 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, 1 MHz. It will be appreciated that the system can be configured to deliver an electric field using a frequency falling within a range, wherein any of the foregoing frequencies can serve as the upper or lower bound of the range, provided that the upper bound is greater than the lower bound.

In some embodiments, the system can be configured to generate one or more applied electric field strengths selected from a range of within 0.25 V/cm to 1000 V/cm, or 0.25 V/cm to 500 V/cm, or 0.25 V/cm to 100 V/cm, or 0.25 V/cm to 50 V/cm. In some embodiments, the system can be configured to generate one or more applied electric field strengths of greater than 3 V/cm. In some embodiments, the system can be configured to generate one or more applied electric field strengths selected from a range of within 1 V/cm to 10 V/cm. In some embodiments, the system can be configured to generate one or more applied electric field strengths selected from a range of within 3 V/cm to 5 V/cm.

In other embodiments, the system can be configured to deliver one or more applied electric field strengths including 0.25 V/cm, 0.5 V/cm, 0.75 V/cm, 1.0 V/cm, 2.0 V/cm, 3.0 V/cm, 5.0 V/cm, 6.0 V/cm, 7.0 V/cm, 8.0 V/cm, 9.0 V/cm, 10.0 V/cm, 20.0 V/cm, 30.0 V/cm, 40.0 V/cm, 50.0 V/cm, 60.0 V/cm, 70.0 V/cm, 80.0 V/cm, 90.0 V/cm, 300.0 V/cm, 125.0 V/cm, 150.0 V/cm, 175.0 V/cm, 400.0 V/cm, 225.0 V/cm, 250.0 V/cm, 275.0 V/cm, 300.0 V/cm, 325.0 V/cm, 350.0 V/cm, 375.0 V/cm, 400.0 V/cm, 425.0 V/cm, 450.0 V/cm, 475.0 V/cm, 500.0 V/cm, 600.0 V/cm, 700.0 V/cm, 800.0 V/cm, 900.0 V/cm, 1000.0 V/cm. It will be appreciated that the system can generate an electric field having a field strength at a treatment site falling within a range, wherein any of the foregoing field strengths can serve as the upper or lower bound of the range, provided that the upper bound is greater than the lower bound.

In some embodiments, an electric field can be applied to the site of a cancerous tumor or tumor resection at a specific frequency or constant frequency range.

In some embodiments, the electric field can be modulated in response to a patient's measured reference or core body temperature and/or a set period of time elapsing. For example, if the patient's reference or core body temperature is higher than a threshold level, the therapy parameters can be modulated to reduce the heat output of the system inside the body. If the patient's reference or core body temperature is higher than a threshold level, the electric field strength can be decreased by 5%, 10%, 15%, 20%, 25%, 30%, 35%, 40%, 45%, 50%, 55%, 60%, 65%, 70%, 75%, 80%, 85%, 90%, 95%, or 100% (e.g., turned off) or can be decreased by an amount falling within a range between any of the foregoing. If the patient's reference or core body temperature is higher than a threshold level, the electric field strength can be decreased by from 5% to 100%, or between 5% and 95%. It will be appreciated that other parameters can also be modulated in order to reduce the amount of heat generated by the system including, for example, current, voltage, and/or frequency.

Alternatively, if the reference or core body temperature is lower than a threshold level, then to maximize exposure to therapeutic electrical fields the electric field strength can be increased. In some embodiments, the electric field strength can be increased by 5%, 10%, 15%, 20%, 25%, 50%, 75%, 100%, 200%, 300%, 500%, 1000% or more, or by an amount falling within a range between any of the foregoing. It will be appreciated that other parameters can also be modulated in order to increase the intensity of the electrical field therapy provided by the system including, for example, current, voltage, and/or frequency.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An implantable field generator device comprising:
a housing; and
a field-generating circuit, wherein the field-generating circuit is disposed within the housing, the field-generating circuit comprising
a circuit ground;
a positive voltage output; and
a negative voltage output;
an equivalent series referencing resistor, wherein the equivalent series referencing resistor is electrically connected in series between the housing and the circuit ground;
a first DC block, AC coupling capacitor;
a first galvanic isolation switch, wherein the first galvanic isolation switch and the first DC block, AC coupling capacitor are connected in series between the positive voltage output and a positive electrode connection terminal;
a second DC block, AC coupling capacitor; and
a second galvanic isolation switch, wherein the second galvanic isolation switch and the second DC block, AC coupling capacitor are connected in series between the negative voltage output and a negative electrode connection terminal.

2. The implantable field generator device of claim 1, wherein the equivalent series referencing resistor has a resistance of 20K ohms or more.

3. The implantable field generator device of claim 1, wherein the equivalent series referencing resistor is configured to limit current through patient contacting therapy electrodes electrically connected with the connection terminals back to the circuit ground in the case of failure of the first DC block, AC coupling capacitor, the second DC block, AC coupling capacitor, the first galvanic isolation switch, or the second galvanic isolation switch.

4. The implantable field generator device of claim 1, wherein the first DC block, AC coupling capacitor, the second DC block, AC coupling capacitor, the first galvanic isolation switch, and the second galvanic isolation switch block DC current and low frequency AC current flow.

5. The implantable field generator device of claim 1, further comprising:
a first therapy electrode, wherein the first therapy electrode is electrically connected with the positive electrode connection terminal; and
a second therapy electrode, wherein the second therapy electrode is electrically connected with the negative electrode connection terminal.

6. The implantable field generator device of claim 5,
wherein the first therapy electrode includes platinum; and
wherein the second therapy electrode includes platinum.

7. The implantable field generator device of claim 1,
wherein the first galvanic isolation switch is an optical isolation switch; and
wherein the second galvanic isolation switch is an optical isolation switch.

8. The implantable field generator device of claim 1, wherein the housing includes a conductive material.

9. An implantable field generator comprising:
a housing;
a voltage supply circuit, wherein the voltage supply circuit is disposed within the housing; and
an H-bridge circuit, wherein the H-bridge circuit is electrically connected with the voltage supply circuit, the H-bridge circuit comprising
a positive voltage output; and
a negative voltage output;
a first bandpass filter;
a first capacitor, wherein the first bandpass filter and the first capacitor are connected in series between the positive voltage output and a first supply electrode connection terminal;
a second bandpass filter;
a second capacitor, wherein the second bandpass filter and the second capacitor are connected in series between the negative voltage output and a second supply electrode connection terminal;
a first resistor;
a second resistor, wherein the first resistor and the second resistor are connected in series between an output of the voltage supply circuit and a ground connected to the voltage supply circuit; and
a conductor, wherein the conductor provides electrical communication between the housing and a point between the first resistor and the second resistor.

10. The implantable field generator of claim 9, further comprising an equivalent series referencing resistor, wherein the equivalent series referencing resistor is connected to the conductor in series between the housing and the point between the first resistor and the second resistor.

11. The implantable field generator of claim 9, wherein the housing includes a conductive material.

12. The implantable field generator of claim 9, wherein the voltage supply circuit has a voltage output of 0 to 12 volts DC.

13. The implantable field generator of claim 9, further comprising:
a first therapy electrode, wherein the first therapy electrode is electrically connected with the first supply electrode connection terminal; and
a second therapy electrode, wherein the second therapy electrode is electrically connected with the second supply electrode connection terminal.

14. An implantable field generator comprising:
a housing;
a voltage supply circuit, wherein the voltage supply circuit is disposed within the housing; and
an H-bridge circuit, wherein the H-bridge circuit is electrically connected with the voltage supply circuit, the H-bridge circuit comprising
a positive voltage output; and
a negative voltage output;
a first LC filter;
a first capacitor, wherein the first LC filter and the first capacitor are connected in series between the positive voltage output and a first supply electrode terminal;
a second LC filter;
a second capacitor, wherein the second LC filter and the second capacitor are connected in series between the negative voltage output and a second supply electrode terminal;
a first resistor;
a second resistor, wherein the first resistor and the second resistor are connected in series between an output of the voltage supply circuit and a ground connected to the voltage supply circuit;
a conductor, wherein the conductor is connected between the housing and a point between the first resistor and the second resistor; and
a two-phase, non-overlapping clock signal generator, wherein the two-phase, non-overlapping clock signal generator is electrically connected with the H-bridge circuit.

15. The implantable field generator of claim 14, further comprising vector selection relays, wherein the vector selection relays are connected in series with a first supply electrode connection terminal and/or a second supply electrode connection terminal.

16. The implantable field generator of claim 15, wherein the vector selection relays are optically coupled solid-state relays.

17. The implantable field generator of claim 14, wherein the housing includes a conductive material.

18. The implantable field generator of claim 14, further comprising:
a first therapy electrode, wherein the first therapy electrode is electrically connected with a first supply electrode connection terminal, the first supply electrode connection terminal electrically connected with the first capacitor; and
a second therapy electrode, wherein the second therapy electrode is electrically connected with a second supply electrode connection terminal, the second supply electrode connection terminal electrically connected with the second capacitor.
